(19) United States
(12) Patent Application Publication
(10) Pub. No.: US 2005/0026154 A1
(43) Pub. Date: Feb. 3, 2005

(54) Masking chemical arrays

(76) Inventors: Laurakay Bruhn, Mountain View, CA (US); Douglas A. Amorese, Los Altos, CA (US); Michael P. Caren, Palo Alto, CA (US); Leslie A. Leonard, Portola Valley, CA (US); Richard J. Pittaro, San Carlos, CA (US); Carol T. Schembri, San Mateo, CA (US); Peter G. Webb, Menlo Park, CA (US); Paul K. Wolber, Los Altos, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/633,609

(22) Filed: Jul. 31, 2003

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

A method of using a chemical array unit having a chemical array with probes at multiple feature locations. A request for test may be read, which test uses a sub-array of the array. A pattern of the sub-array may be retrieved from a memory using the test request, which memory carries a pattern for the sub-array which is retrievable with the different test request. Also, a method of reading a chemical array unit which has been exposed to a sample, and feature locations of which have been rendered incapable of providing signal data representative of binding of a sample component. Further methods, apparatus, and computer program products are provided.

### FIELD OF THE INVENTION

This invention relates to arrays, for example polynucleotide arrays such as DNA arrays, which are useful in diagnostic, screening, gene expression analysis, and other applications.

### BACKGROUND OF THE INVENTION

Chemical arrays such as biopolymer arrays (for example polynucleotide array such as DNA or RNA arrays, or protein arrays), are known and are used, for example, as diagnostic or screening tools. Such arrays include regions of usually different sequence polynucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers onto a substrate, or by in situ synthesis methods. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266, U.S. Pat. No. 6,232,072, U.S. Pat. No. 6,180,351, and U.S. Pat. No. 6,171,797. Other techniques for fabricating biopolymer arrays include known light directed synthesis techniques.

In array fabrication, the probes formed at each feature are usually expensive. Additionally, sample quantities available for testing are usually also very small and it is therefore desirable to simultaneously test the same sample against a large number of different probes on an array. These conditions make it desirable to produce arrays with large numbers of very small (for example, in the range of tens or one or two hundred microns), closely spaced features (for example many thousands of features). After an array has been exposed to a sample, the array is read with a reading apparatus (such as an array “scanner”) which detects the signals (such as a fluorescence pattern) from the array features. Such a reader should typically have a very fine resolution (for example, in the range of five to twenty microns). The signal image resulting from reading the array can then be digitally processed to evaluate which regions (pixels) of read data belong to a given feature as well as the total signal strength from each of the features. The foregoing steps, separately or collectively, are referred to as “feature extraction”. Given the large number of features that are possible on an array, data can be obtained from a sample relating to a great many genes of the organism from which the sample came.

The present invention recognizes that while much of the generated data from reading an array which has been exposed to a sample, has inherent uses in interpreting a state or a response of an organism from which the sample was obtained, it may not relate to a particular inquiry of the array user (for example does the organism exhibit a particular condition of interest). Thus, attempting to interpret all the data derived from an array of many thousands of features may be result in a large amount of data processing irrelevant to the particular inquiry of the array user. Furthermore, where the sample was obtained from a human subject, data may be generated which is irrelevant to the array user's inquiry but which may disclose a state or response of that subject which was never requested and the disclosure of which may raise serious privacy concerns. The present invention recognizes then that it would be desirable to address these issues.

### SUMMARY OF THE INVENTION

The present invention then, provides in one aspect a method of using a chemical array unit having a chemical array with probes at multiple feature locations. The method may include retrieving a pattern of a sub-array from a memory using a test request, which memory carries one or more sub-array patterns for the array each retrievable with a different test request. The method may further include reading the request for a test which uses a sub-array of the array.

A chemical array unit of the type already described may be used by exposing the array to a sample so that sample components can bind to probes at one or more feature locations to provide at each location a detectable signal representative of the binding. In one aspect of the invention some of the feature locations are rendered incapable of providing the detectable signal.

The present invention may also include a method of reading a chemical array unit of a type already described, which array has been exposed to a sample, and in which feature locations have been rendered incapable of providing signal data representative of binding of a sample component. There is further provided by the present invention a method of using a chemical array unit of a type described, which method includes rendering feature locations incapable of providing signal data representative of binding of a sample component in accordance with a predetermined pattern.

In another method of the present invention a sub-array pattern is retrieved from a memory using a test request, which memory carries one or more sub-array patterns for the array each retrievable with a different test request.

Apparatus, computer programs, and computer program products which may execute a method of the present invention, are further provided.

Different embodiments of the present invention may provide any one or more of the following, or other, useful benefits. For example, a simple way may be obtained of identifying data from array features which is relevant to a particular test request of an array user and eliminating, or limiting access to, data which is irrelevant to any test requested. In another example, data which is irrelevant to the array user's inquiry may be relatively simply identified and maintained confidential.

### BRIEF DESCRIPTION OF THE DRAWINGS

Embodiments of the invention will now be described with reference to the following drawings in which:

FIG. 1 illustrates a substrate carrying multiple arrays, such as may be read by a method of the present invention;

FIG. 2 is an enlarged view of a portion of FIG. 2 showing multiple spots or features of one array;

FIG. 3 is an enlarged illustration of a portion of the substrate of FIG. 1;

FIG. 4 illustrates the division of a single array into multiple patterns each of less than all the features of the array and which each may be retrievable from a memory using a pattern indicator such as a test type indicator;

FIG. 5 is a schematic diagram illustrating a user station, reader station, and central data station, all of the present invention, and their interaction;

FIG. 6 illustrates an apparatus of the present invention which can render feature locations of an array incapable of providing signal data representative of binding of a sample component in accordance with a predetermined pattern;

FIG. 7 is similar to FIG. 6 but illustrates one method of operation of the apparatus of FIG. 5;

FIG. 8 is a flowchart illustrating methods of the present invention as performed at a sample collection station and a lab station; and

FIG. 9 is a flowchart illustrating methods of the present invention as performed at an array reader station.

To facilitate understanding, identical reference numerals have been used, where practical, to designate the same elements which are common to different figures. Drawings are not necessarily to scale. Throughout this application any different members of a generic class may have the same reference number followed by different letters (for example, arrays 12a, 12b, 12c, and 12d may generically be referenced as “arrays 12”)

### DETAILED DESCRIPTION OF EMBODIMENTS OF THE INVENTION

Throughout the present application, unless a contrary intention appears, the following terms refer to the indicated characteristics.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes DNA (including cDNA), RNA and oligonucleotides, regardless of the source.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides.

An “oligonucleotide” generally refers to a nucleotide multimer of about 10 to 100 nucleotides in length, while a “polynucleotide” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region. For example, each region may extend into a third dimension in the case where the substrate is porous or where features extend vertically upward, while not having any substantial third dimension measurement (thickness) in the case where the substrate is non-porous. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). An array feature is generally homogenous in composition and concentration and the features may be separated by intervening spaces (although arrays without such separation can be fabricated). In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other).

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

“Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

When one item is indicated as being “remote” from another, this is referenced that the two items are at least in different buildings, and may be at least one mile, ten miles, or at least one hundred miles apart. When different items are indicated as being “local” to each other, they are at least in the same building and may be in the same room of a building. “Communicating”, “transmitting” and the like, reference conveying data representing information as electrical or optical signals over a suitable communication channel (for example, a private or public network, wired, optical fiber, wireless radio or satellite, or otherwise). Any communication or transmission can be between devices which are local or remote from one another. “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something means it is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

When two items are “associated” with one another they are provided in such a way that it is apparent one is related to the other such as where one unambiguously references the other. For example, an array identifier can be associated with an array by being on the array unit (such as on the substrate or housing) that carries the array or on or in a package or kit carrying the array unit. Similarly, a test request can be associated with an array and array identifier by being provided in a same package with them or electronically linked. Another means of association is by means of a common medium (such as paper) carrying both the test request and the array identifier, with the medium being in a same package as the array or with the array identifier also being carried on the array unit. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array. Similarly, an array identifier, test request and the sub-array pattern may be linked in memory by an input of two of them (such as the array identifier and the test request) retrieves the other (such as the sub-array pattern).

A “computer”, “processor” or “processing unit” are used interchangeably and each references any combination of hardware or software which can control components as required to execute recited steps and includes. For example a computer, processor, or processor unit includes a general purpose digital microprocessor suitably programmed to perform all of the steps required of it, or any hardware or software combination which will perform those or equivalent steps. Programming may be accomplished, for example, from a computer readable medium carrying necessary program code (such as a portable storage medium) or by communication from a remote location (such as through a communication channel).

A “memory” or “memory unit” refers to any device which can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “unit” may be the array plus only a substrate on which the array is deposited, although the assembly may be in the form of a package which includes other features (such as a housing with a chamber). “Array unit” may be used interchangeably with “array assembly”.

“Signal data” for a chemical array is data acquired by reading one or multiple features of the array such as in a chemical array reader. This signal data for an array or part of the array (that is, for a pattern of less than all the feature locations such as a sub-array pattern) may be referenced as a “signal image”. A signal image may exist solely as a signal data in a memory but may be presented on a display or some other device for human viewing if desired.

A “package” is one or more items (such as array units optionally with other items) all held together (such as by a common wrapping or protective cover or binding). Normally the common wrapping will also be a protective cover (such as a common wrapping or box) which will provide additional protection to items contained in the package from exposure to the external environment. In the case of just a single array unit a package may be that array unit with some protective covering over the array unit (which protective cover may or may not be an additional part of the array unit itself).

“Sub-array” references a collection of features of the array which are less than all the features of the array (for example, less than 90%, 80%, 60%, 50%, 30%, or 10% of all array features). A “sub-array pattern” is the identification of such features (that is, the pattern in which they are arranged). While features of a sub-array will often be a contiguous set of array features (in the sense that there are no intervening non-sub-array features within the boundaries of the sub-array), this is not necessarily the case and the sub-array pattern can be any arrangement of less than all array features desired. An array may have more than one sub-array patterns, which may or may not overlap with one another. A feature “outside” any sub-array pattern is one which is not a feature of any sub-array pattern.

A “test request” references a type of test which it is desired be performed. The test type may be for testing a sample to ascertain whether it contains certain components quantitatively or qualitatively, such as nucleic acids or peptides or classes of the foregoing, or whether the sample or an organism from which it was derived exhibits a particular condition (for example, the activity of a gene or classes of genes, the presence of particular polymorphisms or class of polymorphisms, or a particular disease condition). A test request can be in any form such as human or machine readable and may or may not actually contain one or more details of the test type itself (for example, the test request may only be an indicator, such as alphanumeric code or other identification of a test type).

When a pattern is “retrieved”, this references that the pattern may be expressly or implicitly retrieved. For example, a pattern of particular feature locations may be retrieved from a memory by expressly retrieving an identification of those feature locations or a boundary (or boundaries) encompassing those feature locations. Alternatively, the pattern of particular features may be implicitly retrieved by retrieving an identification of all feature locations outside the pattern, and the pattern feature locations unambiguously derived from that retrieval as all other feature locations of the array. Express retrieval of sub-array patterns will generally be simpler. In the case of patterns of feature locations that are to be rendered incapable of incapable of providing signal data representative of binding of a sample component, it may often be simpler to retrieve these implicitly by retrieving all desired sub-array patterns then deriving the pattern of the features to be rendered incapable as all other array feature locations which are outside any retrieved sub-array pattern.

It will also be appreciated that throughout the present application, that words such as “front”, “back”, “top”, “upper”, and “lower” are used in a relative sense only.

“May” refers to optionally.

Any recited method can be carried out in the order of events recited or in any other order which is logically possible. Reference to a singular item, includes the possibility that there are plural of the same item present. All patents and other references cited in this application, are incorporated into this application by reference except insofar as anything in those patents or references, including definitions, conflicts with anything in the present application (in which case the present application is to prevail).

Methods of using arrays in accordance with the present invention may further include reading an array identifier associated with the chemical array unit (such as by being carried thereon). In this case the sub-array pattern may be retrieved from the memory using both the array identifier and test request (which may also be associated with the array). The memory in this situation may carry multiple sub-array patterns for one or more arrays, each pattern retrievable with a different combination of array identifier and test request.

One particular use is reading the array where the array has been exposed to a sample. In this case the method may include acquiring and saving signal data representative of binding of a sample component from feature locations based on one or more retrieved sub-array patterns. For example, such signal data may be acquired and saved from only feature locations of the one or more retrieved sub-array patterns. Alternatively or additionally, the method may include applying a same signal processing method to acquired signal data representative of binding of a sample component from feature locations based on one or more retrieved sub-array patterns. For example, the method may include applying a same signal processing method only to acquired signal data from feature locations of one or more retrieved sub-array patterns. Note that this alternative or additional procedure does not prevent signal processing methods being applied to signal data acquired from all array feature locations, where there is a processing method which is applied to signal data from array feature locations based on the one or more retrieved sub-array patterns. Also, in any embodiment herein the referenced signal data from feature locations which is representative of binding of a sample component to those locations, will often be data which is representative of binding of a sample component whose presence or amount in the sample is unknown prior to reading the array (for example, not a component known to bind to an array probe that was intentionally added to the sample as a reference target for that probe).

It is also possible in the present invention that multiple test type requests are used. In this situations the method may include reading an array identifier and the test requests, all associated with the array. Multiple sub-array patterns may be retrieved from a memory using both the array identifier and the test requests. Such a memory may carry multiple sub-array patterns for each of multiple arrays, each sub-array pattern retrievable with a different combination of array identifier and pattern indicator.

In some methods of the present invention, feature locations of the sub-array pattern may be selected as a result of feature locations outside any sub-array pattern being physically masked. For example, feature locations outside any sub-array pattern may be incapable of providing signal data representative of binding of a sample component. There are various ways this incapacity may occur. For example, it may be the result of binding of a sample component to such outside features having been prevented, or as a result of having an excess of a label on those features (such as a fluorescent label linked to sample components), or such outside features having a material thereon which prevents reading of signal data representative of binding of a sample component (for example, dried salts, specific binding agents such as other oligonucleotides or antibodies, or other material which blocks or otherwise prevents reading of signal from a fluorescent label at a feature). An “excess” in this context references label on a feature location which is not there as a result of sample. In this situation such a feature location may produce a signal at least 80%, 90%, 100%, 120%, 200% or at least 300% the maximum signal that is produced by any feature location of the array as a result of a probe at that location having bound to a sample component. In another example the incapacity may be the result of probes at the incapable feature locations having been damaged to prevent binding (such as by cross-linking or cleaving of the probes at those locations). In a case where signal data of feature locations within a sub-array pattern is acquired from a label at those feature locations, the incapacity of non-feature locations may also be as a result of the label thereon having been damaged to prevent signal data being obtained from the label (such as by bleaching of a fluorescent or chemiluminescent label).

Masking may also be the result of not acquiring a signal from feature locations outside any or all retrieved sub-array patterns (that is, signal data may be acquired only from features of the retrieved sub-array patterns). For example, signal data may be acquired from feature locations of each sub-array by illuminating those locations with an interrogating light and detecting any light emitted in response to the interrogating light. No signal data representative of binding of a sample component is acquired from feature locations outside any or all retrieved sub-array patterns as a result of not illuminating such feature locations with the interrogating light.

In other methods of the present invention, feature locations of one or more sub-arrays may be selected as a result of feature locations outside such sub-arrays being masked during data processing. For example, in one such masking technique signal data may be acquired from both the one or more sub-array feature locations as well as feature locations outside the one or more sub-arrays. However, acquired signal data from the sub-array feature locations is saved in a memory while acquired signal data for feature locations outside any or all retrieved sub-arrays is not saved in the memory. Note that this technique allows for all acquired signal data to be temporarily saved in a memory (for example, a volatile memory) while only the signal data from retrieved sub-arrays features is saved in another memory (for example, a more permanent non-volatile memory). Optionally, one could of course encrypt the data that is saved (for example, with a suitable algorithm and encryption key). In another such masking technique the method includes applying a same signal processing method only to acquired signal data from features of one or more retrieved sub-array patterns (for example, a different signal processing technique or no signal processing technique may be applied to features outside any or all retrieved sub-array patterns). One example of the foregoing is where the same signal processing method includes an encryption method based on a key, in which case the method may additionally include applying an encryption method based on a different key to signal data acquired from features outside any or all retrieved sub-array patterns. A second example is applying different signal processing methods to acquired signal data from features of different retrieved sub-array patterns. In this second example results from the application of such different signal processing methods may be independent such that a result from one sub-array cannot be derived from a result from one or more other sub-arrays. Furthermore, such results from applying the different signal processing methods may be forwarded to different locations.

Further, some such results from applying different signal processing methods may be rejected or accepted based on a comparison of those results (that is, with one another) or a comparison of a characteristic of the feature locations in the different sub-arrays (for example, results from sub-arrays having a higher proportion of feature locations producing a weak signal may be rejected). Another comparison may be a voting system where different algorithms (or the same algorithms with different parameters) are applied to different sub-arrays, and a condition that a majority of the algorithms diagnose or determine would be considered the proper result. In methods of the present invention the array may have been exposed to a sample obtained from an individual, in which case the sub-array pattern may be retrieved also using an identification of the individual. For example, where a test request is for a test the results of which are dependent upon known genetic polymorphisms and the array contains features for the different polymorphic variants of one or more genes, different sub-array patterns may be retrieved each with probes for the different variants depending upon the identity of the individual (for example, racial characteristics or a unique identifier for that individual which can be used to retrieve information stored in a database on which variants are relevant to that person).

In methods of the present invention signal data may be acquired from feature locations which have not been rendered incapable of providing signal data representative of binding a sample component. Such feature locations may be less than all the array feature locations (for example, feature locations outside any or all retrieved sub-array patterns). Signal may be acquired from a label at feature locations, in which case the rendering incapable may include damaging a label to prevent signal data being obtained from the label (such as by bleaching a label as mentioned above). Other methods of the rendering include selectively preventing binding of a sample component to probes at feature locations, such as by activating heating elements at some of the feature locations, or providing an excess of the label at those features. In one embodiment both the rendering and the acquiring may be executed in a same apparatus, optionally while the array unit remains seated in a same holder (for example, a holder in an array reader which uses interrogating light to read the array and bleach features). Note that the rendering may be performed before, during, or after exposing the array to the sample.

Other methods of the present invention may include retrieving a pattern of less than all feature locations (such as a sub-array pattern) of a chemical array from a memory using a test request and optionally also an array identifier. The memory may carry multiple sub-array patterns for each of one or more arrays each retrievable with a different test request (for example, each retrievable with a different combination of array identifier and test request). The array identifier and test request may both be received from a remote location (such as an array user station or reader station), and the retrieved pattern of less than all the array feature locations may be communicated to the remote location in response to the received test request and any received array identifier.

As mentioned above, methods of using a chemical array are provided in which a predetermined pattern of feature locations is rendered incapable of providing signal data representative of binding of a sample component. Such a predetermined pattern will typically be some features less than all the features (for example less than 80%, 60% or 30% of all features), but could be all features if the array is read as needed before the rendering incapable.

An apparatus of the present invention may simply include a processor to execute any one or more methods as described herein. One type of apparatus of the present invention may also include an interrogating source (such as a light source to illuminate array feature locations with an interrogating light, which light source may or may not be the same as a light source of a deactivator). A detector to detect light emitted in response to the interrogating light may be further included along with a processor which causes the apparatus to execute a method of the present invention. Another type of apparatus of the present invention may instead have a deactivator (for example, a power supply for heating elements for each of multiple feature locations) which renders feature locations incapable of providing signal data representative of binding of a sample component, and a processor controlling the deactivator so as to execute a method of the present invention (for example, by controlling the power supply to deliver power to selected heating elements at array feature locations in accordance with the a pattern).

Computer program products of the present invention may include a computer readable medium (such as a memory) carrying a computer program which when loaded into a computer executes a method described herein.

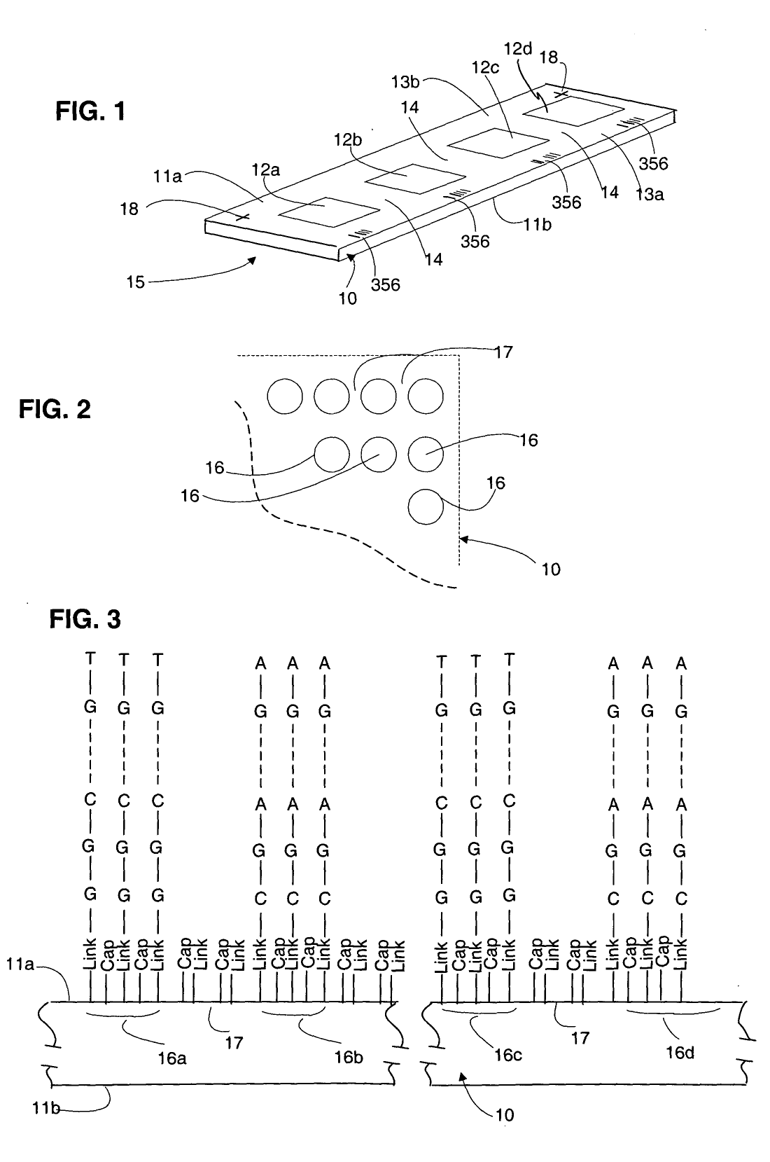

Referring now to FIGS. 1-3, an array assembly 15 (which may also be referenced as an “array unit”) which can be used in methods and apparatus of the present invention, includes arrays 12 which may be read to obtain an array signal image used in methods of the present invention. Substrate 10 may also be in the form of an a rigid substrate 10 (for example, a transparent non-porous material such as glass or silica) of limited length, carrying one or more arrays 12 disposed along a front surface 11a of substrate 10 and separated by inter-array areas 14. Alternatively, substrate 10 can be flexible (such as a flexible web). The substrate may be of one material or of multi-layer construction. Substrate 10 is typically non-porous, and may be smooth and planar, or have irregularities, such as depressions or elevations (although irregular substrate surfaces may make reading of the exposed array more difficult). However, even a flat planar substrate 10 may have small irregularities in its shape (for example, front side 11a may be slightly bent or bowed). A back side 11b of substrate 10 does not carry any arrays 12. The arrays on substrate 10 can be designed for testing against any type of sample, whether: a trial sample; reference sample; a combination of the foregoing; or a known mixture of polynucleotides, proteins, polysaccharides and the like (in which case the arrays may be composed of features carrying unknown sequences to be evaluated). While four arrays 12 are shown in FIG. 1, it will be understood that substrate 10 may use any number of desired arrays 12 such as at least one, two, five, ten, twenty, fifty, or one hundred (or even at least five hundred, one thousand, or at least three thousand). When more than one array 12 is present they may be arranged end to end along the lengthwise direction of substrate 10. Depending upon intended use, any or all of arrays 12 may be the same or different from one another and each will contain multiple spots or features 16 of biopolymers in the form of polynucleotides.

A typical array 12 may contain more than: ten, one hundred, one thousand, or ten thousand features. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature of the same composition are excluded, the remaining features may account for at least 5%, 10%, or 20% of the total number of features). The features may have a maximum dimension of between 20 (or 50) to 100 (or 80) microns and be spaced apart by less than 130 microns (or by less than 100 or 50 microns). Various feature densities on the substrate surface are possible. For example, features having a maximum dimension greater than any of the foregoing figures may be present on the surface of at least 30 features/mm², 40 features/mm², or 60 features/mm². While round features 16 are shown, various other feature shapes are possible (such as elliptical). The features 16 may also be arranged in other configurations (for example, circular) rather than the rectilinear grid illustrated. Similarly, arrays 12 on a same substrate 10 need not be laid out in a linear configuration.

Each array 12 may cover an area of less than 100 cm², or even less than 50 cm², 10 cm² or 1 cm². In many embodiments, particularly when substrate 10 is rigid, it may be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. When substrate 10 is flexible, it may be of various lengths including at least 1 m, at least 2 m, or at least 5 m (or even at least 10 m). With arrays that are read by detecting fluorescence, the substrate 10 may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

In the case where arrays 12 are formed by the conventional in situ or deposition of previously obtained moieties, as described above, by depositing for each feature a droplet of reagent in each cycle such as by using a pulse jet such as an inkjet type head, interfeature areas 17 will typically be present which do not carry any polynucleotide. It will be appreciated though, that the interfeature areas 17 could be of various sizes and configurations. Further, such interfeature areas 17 need not be present at all (such as when arrays are fabricated using light directed synthesis techniques). Where interfeature areas 17 are present, the features 16 may be spaced apart by a distance greater than 0 and less than 70%, 60% 50%, 25%, or 10% of a maximum dimension of the feature. Each feature 16 carries a predetermined polynucleotide (which includes the possibility of mixtures of polynucleotides). As per usual, A, C, G, T represent the usual four nucleotides. “Link” (see FIG. 3 in particular) represents a linking agent (molecule) covalently bound to the front surface and a first nucleotide, as provided by a method of the present invention and as further described below. The Link serves to functionalize the surface for binding by the first nucleotide during the in situ process. “Cap” represents a capping agent. The Link may be any of the “second silanes” referenced in U.S. Pat. No. 6,444,268 while the Cap may be any of the “first silanes” in that patent. However, different linking layer compositions than those silanes could be used. As already mentioned, the foregoing patents are incorporated herein by reference, including for example the details of the linking layer compositions used therein.

Substrate 10 also has one or more array identifiers 356 each in the form of a bar code. Identifiers 356 may be associated with an array by being: directly printed onto the substrate 10 or a housing (not shown) carrying substrate 10; printed onto labels attached to substrate 10 or a housing carrying substrate 10; contained in a memory (for example, a solid state memory) attached to substrate 10 or a housing carrying substrate 10; or be provided on a printed label or paper or some other medium or in a memory, any of which is received in or on a same package containing the array unit 15 (and therefore also containing substrate 10). Identifiers such as other optical or magnetic identifiers could be used instead of bar codes, and which will carry the information discussed below. Each array identifier 356 may be associated with its corresponding array by being positioned adjacent that array 12 on the same substrate 10. However, this need not be the case and array identifiers 356 can be positioned elsewhere on substrate 10 if some other means of associating each identifier 356 with its corresponding array 12 is provided (for example, by relative physical locations). Further, a single identifier might be provided which is associated with more than one array 12 on a same substrate 10 and such one or more identifiers may be positioned on a leading or trailing end of substrate 10. Each identifier 356 may also be associated with an array by being in or on a same package or kit which contained by the array and is received by a user. The substrate may further have one or more fiducial marks 18 for alignment purposes during array fabrication or reading.

FIGS. 2 and 3 illustrate ideal features 16 of an array 12 where the actual features formed are the same as the target (or “aim”) features, with each feature 16 being uniform in shape, size and composition, and the features being regularly spaced. Such ideally shaped features may not always be possible to obtain but this is not critical in any event. Suitable drop deposition methods for fabricating arrays 12 include those as described in U.S. Pat. No. 6,180,351, U.S. Pat. No. 6,242,266, U.S. Pat. No. 6,306,599, and U.S. Pat. No. 6,420,180. As mentioned above, the foregoing references are incorporated herein by reference particularly as relates to the in situ fabrication apparatus and methods disclosed therein. Alternatively, arrays 12 can be fabricated by known light directed synthesis methods.

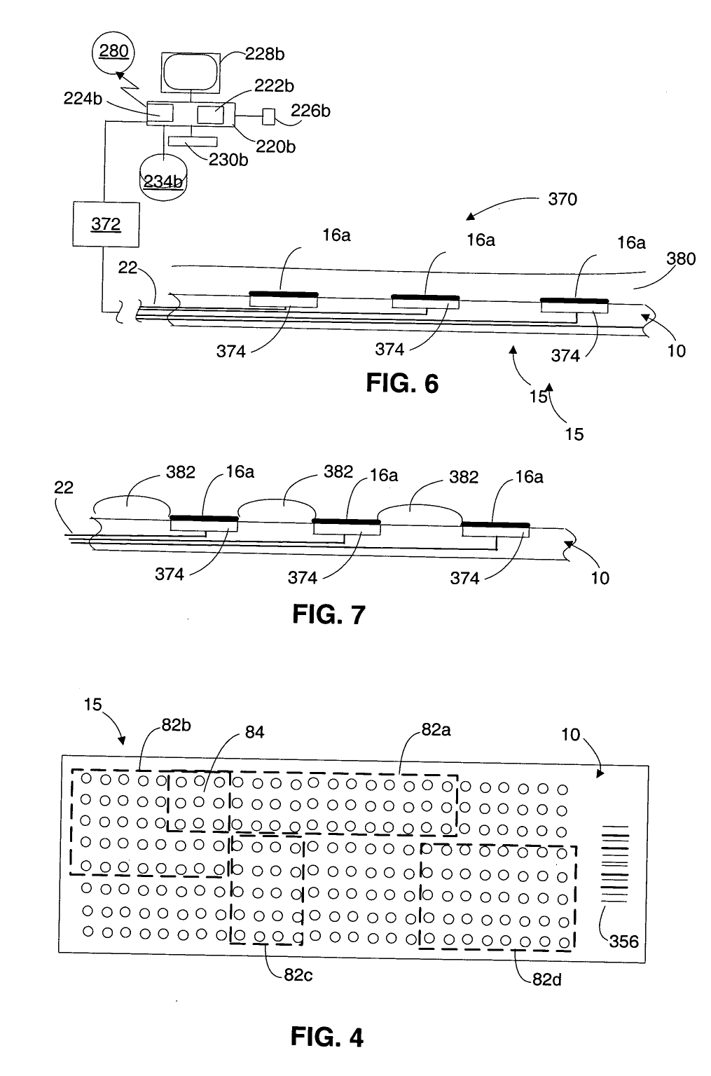

FIG. 4 shows an array unit 15 carrying a single array 12 and illustrates multiple sub-array patterns 82a through 82d each consisting of features 16 within the boundaries of each pattern 82 shown. Each such pattern 82 will include features which are useful for at least one test, for example a test for expression level of certain genes or a class of genes, a test for gene polymorphisms, a test for copy number of a gene or class of genes, or a test for the presence of a pathogen.

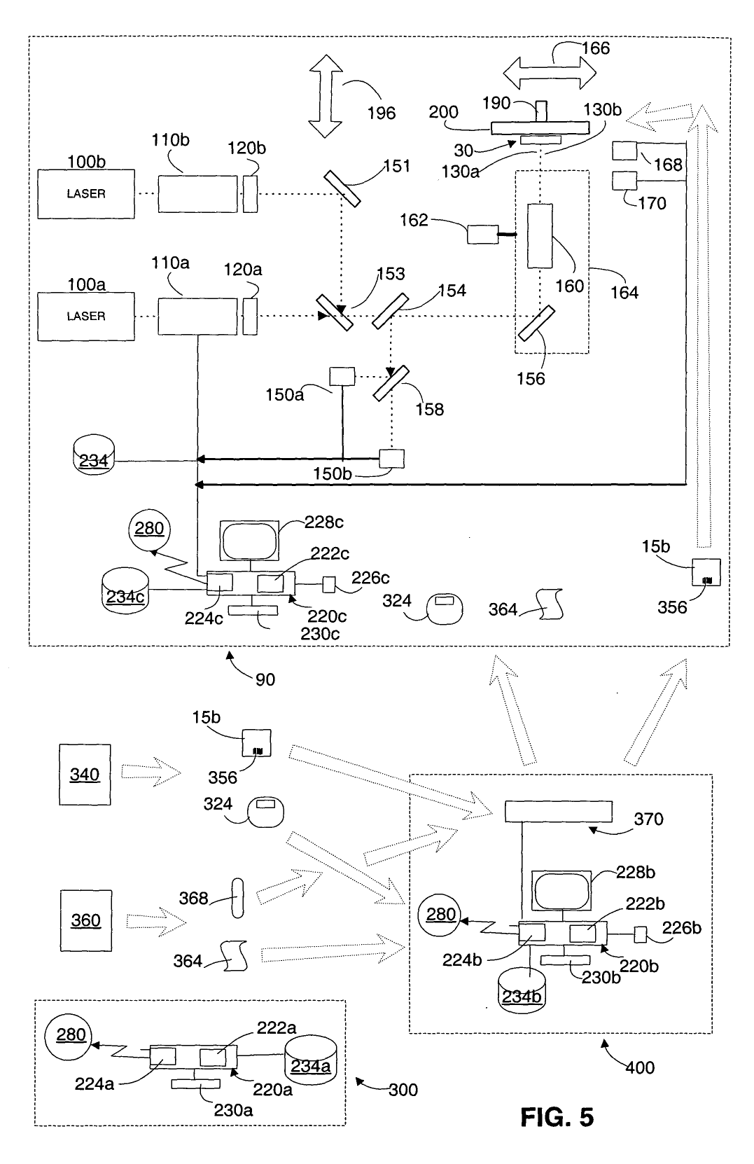

The actual patterns 82 (in this case the boundaries defining each sub-array) are not visible on array 12 in FIG. 4, but instead are stored as boundary location data in a memory 234a of a central data station 300 (see FIG. 5) each linked with a different test request and all linked with the array identifier 356 of FIG. 4. Memory 234a will typically store sub-array patterns for each of multiple different arrays having different array layouts, the sub-array patterns for each array each linked with a different test request and all linked with the identifier for that array. In this way each sub-array pattern 82 (or saved sub-array pattern for any other array) can be retrieved from the memory 234a with a different combination of the array identifier and test request. Referring to FIG. 5, central data station 300 also includes a processor 220a which has access to memory 234a and a communication module 224a through which it may communicate with a remote site through a communication channel 280 (such as a network, for example the internet, a telephone network, a WAN or LAN, or satellite link). Processor 220a also has access to a media reader/writer 222 which can read and write to a removable portable memory 324 (such as a magnetic or optical disk, or solid state memory) and may receive operator input through input device 230a (which may be a keyboard, mouse, voice command module, or other devices). In an alternative arrangement, all the sub-array patterns for a given array and their linked test requests and array identifier can be saved in, and retrieved from, portable memory 324. In any event, such information can be stored in memory 234a or portable memory 324 either at the time of fabrication of an array 12 or later (for example, it may be learned later that new sub-array patterns are useful for additional different tests). Data station 300 is “central” in the sense that it may receive requests for sub-array patterns from many remote and/or local (that is, non-remote) locations. Data station 300 may or may not be located at or local to an array fabrication station.

Continuing to refer to FIG. 5, a user station 400 is shown which is provided with a sample exposure apparatus in the form of sample exposure unit 370 controlled by a processor 220b. Processor 220b has access to various components of a same type as described in connection with central data station 300 (these same component types being numbered the same for stations 300, 400 except with an “a” or a “b”). Processor 220b also has access to a display 228b and a machine reader 226b which reads an identifier 356 from an array unit 15 and provides the read identifier to processor 220b. When identifier 356 is in the form of a bar code, that reader 226b may be a suitable bar code reader.

Sample exposure unit 370 provides a location or station at which a sample may be exposed to an array to allow binding of one or more components therein to array features. Exposure unit 370 may include deactivator in the form of a power supply 372 connectable to a particular type of array unit 15 shown in FIG. 6. Array unit 15 of FIG. 6 is similar in construction to array units 15 of FIGS. 1-4 but additionally includes heating elements 374 immediately adjacent front surface 11a of substrate 10 at each location of features 16. Power supply 372 may be controlled by processor 220b so as deliver power to selected heating elements 374 in accordance with a pattern directed by processor 220b. Such a pattern may be a pattern of any or all array features 16 which are outside any or all sub-array patterns for an array 12 of an array unit 15b received at station 400. These sub-array patterns may be retrieved by processor 220b from remote memory 224a through communication channel 280, using one or more test requests on a medium 364 also received at station 400 (and read in reader/writer 222b) and the array identifier 356 of the array 12 of received array unit 15b (read by reader 226b). As mentioned previously, the retrieval may also use an identification of a source of the sample such as an identification of an individual from which the sample was obtained. Test requests received on medium 364 can either be read by reader 226c (if the test requests are of a type suitable for such reading, for example a bar code) or read by an operator at station 90 and manually input by her through input device 230c. Alternatively, sub-array patterns may be retrieved from a portable memory 324 received at station 400 in association with array unit 15b, using the one or more test requests and array identifier received as in the foregoing manner. Processor 220b causes deactivator 372 to render features incapable of providing signal data representative of binding of a sample component in accordance with a predetermined pattern (such as those features outside any or all retrieved sub-array patterns), in any of several ways. These include ways which can be used before a sample 380 in FIG. 6 is exposed to the array 12. For example, sufficient power could be applied to damage probes at features 16 as a result of cross-linking or cleavage from substrate 10, or other mechanisms. Other ways can be used during exposure of array 12 to sample 380 (specifically during the binding or hybridization of sample components to features). For example, sufficient power can be provided to heating elements 374 at feature locations of the predetermined pattern to selectively prevent binding of a component of sample 380 to probes at those feature locations. In another example where a detectable signal is provided by a label which is bound to feature locations at which a sample component is bound to probes, the rendering may include providing an excess of the label at those features by activating heating elements 374 at those features to evaporate sample 380 at those locations as illustrated in FIG. 7. Since sample 380 in this case contains a large amount of label, this will provide an excess of the labeled material at those locations. However, care should be taken not to extensively wash the array to the point where the excess is washed away.

The apparatus in FIG. 5 further illustrates an array reader station 90. Reader station 90 may sometimes be referenced as an array “scanner”. In FIG. 4, a light system provides coherent light from a laser 100 which passes through an electro-optic modulator (EOM) 110 with attached polarizer 120. Each laser 100a, 100b may be of different wavelength (for example, laser 100a providing red light with a peak emission at 630 nm, and laser 100b providing green light with a peak emission at 530 nm) and each has its own corresponding EOM 110a, 110b and polarizer 120a, 120b. The resulting light beams are coherent and monochromatic.

The red interrogating light beam originating from laser 100a is directed along path 130a while the interrogating green beam originating from laser 100b is directed along respective paths 130b. Light is directed along all of the paths 130a, 130b by means of full mirror 151, dichroic mirror 153, and full mirror 156 onto two different locations of an array being read (namely an array 12 of an array unit 15 mounted on holder 200), using optical components in beam focuser 160. Note though that FIG. 5 shows the paths 130a, 130b of the two beams as being coincident up until the position of a mirror 158, for the sake of simplicity. The angle of separation of the beams may be such that each interrogating light beam is directed along a corresponding path 130a, 130b toward front surface 11a at an angle equal that is greater than or equal to 0 degrees and up to 45 degrees to a normal to the back surface (for example less than 1 degree, such as 0.5 degrees). Such an arrangement allows the two interrogating light beams to pass through the same optical system while reducing saturation of fluorescent labels at features 16 as well as channel cross-talk. A control signal in the form of a variable voltage applied to each corresponding EOM 110a, 110b by a processor 220c, changes the polarization of the exiting light which is thus more or less attenuated by the corresponding polarizer 120a, 120b. Processor 220c has access to components of a type already described in connection with 220b of station 400 (and such components are numbered the same but with a “c” rather than a “b”). Thus, each EOM 110 and corresponding polarizer 120 together act as a variable optical attenuator which can alter the power density of the light exiting from the attenuator. Hence each EOM 110 alters the power density of the interrogating light spot originating from one of lasers.

Each of the two beams provided on paths 130a, 130b then provide two spatially separated spots on an array 12 of an array unit 15 mounted on holder 200. These may be focused on front surface 11a directly without passing through substrate 10 when the array is being read with front surface 11a facing beam focuser 160 (that is, facing down in FIG. 4), or may be focused on front surface 11a after first passing through substrate 10 when the array is being read with front surface 11a facing away from beam focuser 160 (that is, facing up in FIG. 4). Various patterns for the spot separation can be used but the pattern of spots relative to one another will generally remain fixed unless independent optics were provided for the different beam paths 130. Note also that with the foregoing configuration the longer wavelength red light will generally be positioned to illuminate a given region of a feature before a spot of the shorter green light also tending to reduce triplet saturation as described in U.S. Pat. No. 6,320,196. As already mentioned, that patent is incorporated herein by reference in relation to the reading methods described therein.

Light emitted, in particular fluorescence, at two different wavelengths (for example, green and red light) from regions illuminated by the green and red interrogating light spots, in response to the interrogating light, is imaged using the same optics in focuser/scanner 160, and is reflected off mirror 156 and dichroic 154. The two different wavelengths are separated by a further dichroic mirror 158. There will be two paths of detection resulting from the spaced two interrogating light spots. As already mentioned though, for the sake of clarity these are only shown as one path in FIG. 5 up until mirror 158. Dichroic mirror 158 will direct red fluorescent light resulting from one interrogating light spot onto a detector 150a, while green fluorescent light resulting from another interrogating light spot will be directed onto detector 150b. More optical components (not shown) may be used between the dichroic and each of the two detectors 150 (such as lenses, pinholes, filters, fibers etc.) and each detector 150 may be of various different types (e.g. a photo-multiplier tube (PMT) or a CCD or an avalanche photodiode (APD)). All of the optical components through which light emitted from an array 12 in response to the illuminating laser light, passes to the two detectors 150, together with those detectors, form a detection system. This detection system has a fixed focal plane on the array 12 being read for a given position of the autofocus system (that is, in direction 196).

Instead of using dichroic 158, one can also use a design that images the different scanning spots onto different light-guiding fibers that then guide the signal from each one of these to a different detector. Such an arrangement for two scanning spots is described in U.S. Pat. No. 6,320,196.

In order to raster scan red and green interrogating light spots, the scanner is provided with a scan system. In this manner, each of the multiple features 16 of the array is read, with each read feature containing multiple pixels (for example, more than five, ten, or twenty). This can be accomplished by providing a housing 164 containing mirror 158 and focuser 160, which housing 164 can be moved in a first direction along a line (that is, from left to right or the reverse as viewed in FIG. 5) by a transporter 162. The second direction 192 of scanning (line transitioning) can be provided by second transporter which may include a motor and lead screw or belt (not shown) to move holder 200 along one or more tracks. The second transporter may use a same or different actuator components to accomplish coarse (a larger number of lines) movement and finer movement (a smaller number of lines). Of course, other scanning patterns could be used.

An autofocus detector 170 is also provided to sense any offset between different locations on array 12 when in the reading position, and a determined position of the focal plane of the detection system. An autofocus system includes detector 170, processor 220, and a motorized adjuster to move holder in the direction of arrow 196 (which may be referenced as a “z-axis” direction). A suitable chemical array autofocus system is described in U.S. Pat. No. 6,486,457.

Processor 220c of the apparatus is connected to receive signals from the detectors 150a, 150b. Each detector is part of another detection “channel”. The signals in each channel are obtained at each of the two detected wavelengths from emitted light for each scanned pixel on array 12 when at the reading position mounted in holder 200. Processor 220c also receives the signal from autofocus offset detector 170, and provides the control signal to EOM 110, and controls the scan system. Processor 220c may also analyze, store, and/or output data relating to emitted signals received from detectors 150a, 150b in a known manner, as well as control the sensitivities of one or more of the four detectors.

Additionally processor 220c may retrieve one or more sub-array patterns for an array 12 of an array unit 15b received at reader station 90. These sub-array patterns may be retrieved by processor 220c from remote memory 224a through communication channel 280, using one or more test requests on a medium 364 also received at station 90 (and read in reader/writer 222c) and the array identifier 356 of the array 12 of received array unit 15b (read by reader 226c). Test requests received on medium 364 can either be read by reader 226c (if the test requests are of a type suitable for such reading, for example a bar code) or read by an operator at station 90 and manually input by her through input device 230c. Alternatively, sub-array patterns may be retrieved from a portable memory 324 received at station 90 in association with array unit 15b, using the one or more test requests and array identifier received as in the foregoing manner.

Sub-array patterns retrieved by processor 220c may be used so that signal data from an array being read at reader station 90 is acquired and saved from feature locations based on one or more retrieved sub-array patterns. This can be accomplished by controlling EOMs 110 so as to only illuminate feature locations of one or more retrieved sub-array patterns. Alternatively, all features of the array being read can be illuminated but processor 220c discards all feature locations outside the one or more (or all) retrieved sub-array patterns and only saves in memory 234c the data from feature locations within one or more (or all) of the retrieved sub-arrays. In an alternative embodiment, signal data from all feature locations of an array being read at reader station 90 may be acquired and saved. However, a same signal processing method may be applied only to acquired signal data representative of binding of a sample component, from feature locations of one or more retrieved sub-array patterns retrieved by processor 220c, as described further below. Of course, other methods may be used to acquire and save signal data representative of binding of a sample component from only feature locations of one or more retrieved sub-array patterns. Such other methods include, for feature locations outside any or all retrieved sub-array patterns, blocking light emitted from those outside features, modulating gain of a detector or detector circuit (for example, decreasing such gain to about zero for detected light from such outside feature locations), turning off a digitizer which may be part of the detector circuitry, or adding zeros to digitized detected signal from such outside features.

Reader station may also have the ability to render feature locations outside any or all retrieved sub-array patterns, incapable of producing signal data representative of sample component binding. This can be done by processor 220c predetermining a pattern of all such feature locations using the retrieved sub-array patterns and selectively bleaching all feature locations of the predetermined pattern by controlling EOM 110b and/or laser 100b to deliver sufficient power to such feature locations to bleach any fluorescent label there.

The components of the reader station 90 may all be contained within the same housing of a single same apparatus, or processor 220c and devices 222c through 230c may be a separate unit such as a standalone computer with the appropriate peripherals. One particular reader station is disclosed in U.S. Pat. No. 6,406,849. Another particular reader station that may be used is the AGILENT MICROARRAY SCANNER manufactured by Agilent Technologies, Palo Alto, Calif.

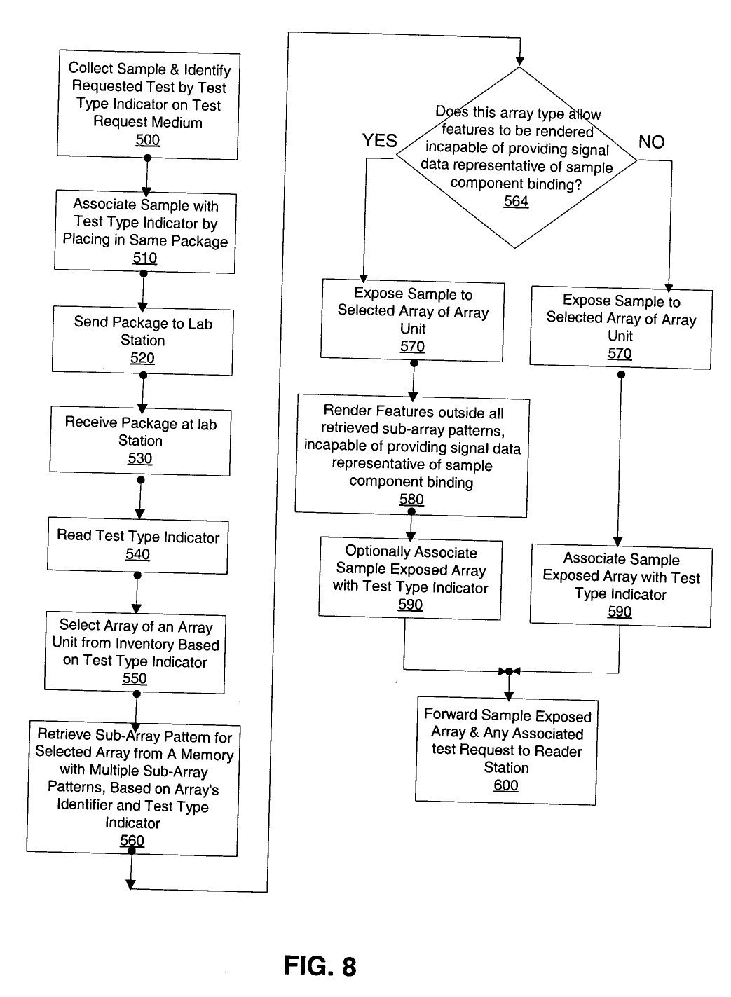

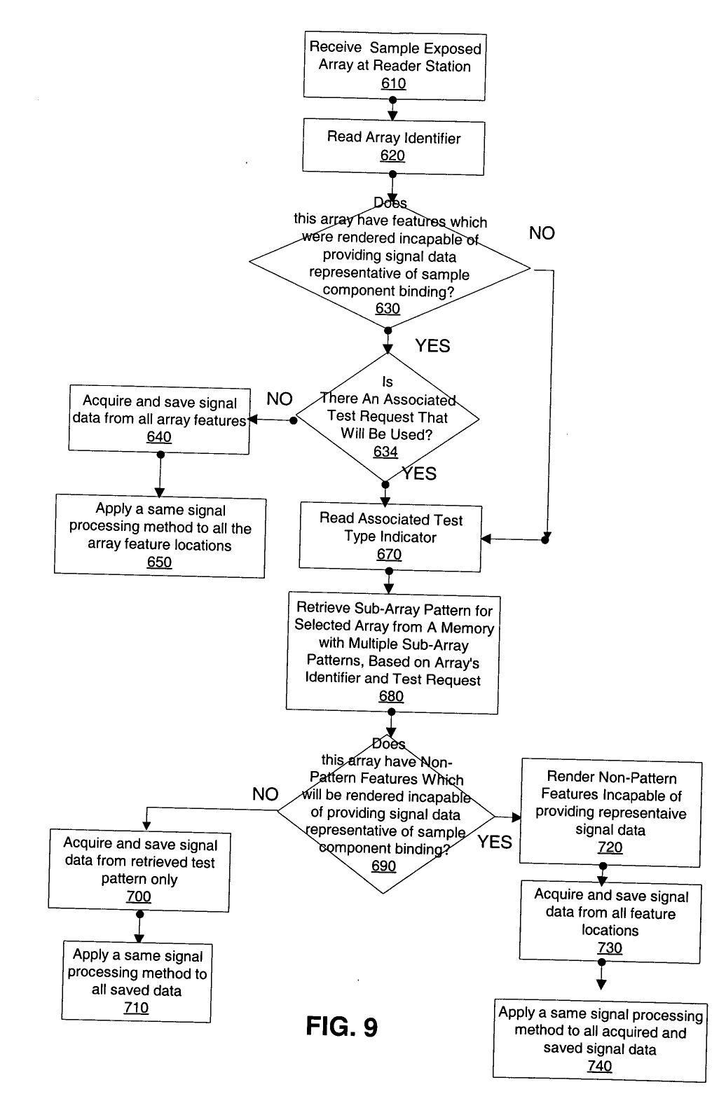

One mode of operation of methods of the present invention will now be described with particular reference to the flowcharts of FIGS. 8 and 9. Reference numerals in parentheses refer to events shown in FIGS. 8 and 9. It will be presumed that different arrays have already been fabricated, various tests for sub-arrays of different arrays identified, and this information along with linked array identifiers and test requests for those tests saved in memory 234a such that each sub-array can be retrieved from memory 234a with a different combination of array identifier and test request. Alternatively, as previously mentioned such information for each array unit 15 can be stored on a portable storage medium 324. It will also be assumed that these test requests are known to individuals who might wish one or more such tests, such as a result of the test types being of common descriptors in a research lab, clinical lab, or doctor's office, or elsewhere or such information otherwise being made available to those locations (through publications, advertisements, internet, and the like). Multiple packages 340 containing an fabricated array units 15b and any associated portable storage medium 324 (associated as a result of being in a same package) may have already been provided to user station 400 and stored there as part of an inventory (each received array and any associated storage medium being kept in association by being stored together, such as in package 340). Alternatively, a package 340 with a particular array unit 15 could be ordered by user station 400 in response to receiving a particular test request.

First, an individual at a research lab, clinical lab, doctor's office, or elsewhere, collects a sample (500) from an individual or other source in a sample container 368. The test or tests which that individual desires to have performed on the sample are recorded (500) as one or more test requests on test request medium 364 which may be a piece of paper or order form, or portable memory. The test requests may simply be written as to the type of test desired or may be a reference to a test identifier (such as a unique code). The individual may additionally include on medium 364 an identification of a source of the sample (such as an individual patient's identification, for example Social Security Number, patient name, and the like). Sample container 368 is then associated (510) with medium 364 by being packaged together in a same package 360 which is forwarded (520) to user station 400.

At user station 400 package 360 is received (530) and the one or more test requests read (540) by reader 226b or by an operator and manually input for access by processor 220b by input device 230b. One or more arrays required to perform the requested tests are then selected (550) by processor 220b from inventory based on the read test requests (or such arrays may be ordered automatically by processor 220b on an as needed basis). The required arrays can be selected by reference to a list of test type indicators and array identifiers of arrays to be used for those tests previously stored in memory 234b. The patterns of one or more sub-arrays of the selected array(s) are then retrieved (560) over communication channel 280 by processor 220b from memory 234a, based on the array identifier 356 of a selected array (which may be read by reader 226b or read from the list previously mentioned). For each selected array it is then determined (564) if the array is of a type which allows features to be rendered incapable at the user station 400, of providing signal data representative of sample component binding (for example, of a type as shown in FIGS. 6 and 7). This determination can be made by an operator by visually inspecting the array unit 15b of a retrieved array, or by the processor from the list previously referenced if that list includes such information.

If the answer to the determination (564) is YES or NO, then the sample in container 368 is exposed (570) to the one or more selected arrays to allow binding of sample components thereto. If the sample is a liquid sample it may be used as is (with or without further preparation depending upon the composition of the received sample) or it may be prepared as a suitable liquid sample (for example, a liquid aqueous sample) for exposure to the array. Samples can be prepared for exposure to an array 15 using methods such as described in U.S. Pat. No. 6,235,483 or 6,132,997. Samples could also be checked for quality prior to exposing them to an array (for example, immediately prior to event 570 or elsewhere) and only exposed to the array if the quality check is passed (this could also be considered a YES/NO determination). Quality checks may include sample degradation (physical or chemical), or contamination (for example, for any foreign organism or inappropriate cells). Sample preparation may, for example, provide fluorescent labels attached to sample components so that features of an array to which sample components bind, will produce a fluorescence signal in response to an interrogating light. After a suitable time of exposure, the array may then be washed with buffer then water, and dried following washing then inserted into a scanner for reading in a manner already described. Suitable conditions for such binding, for example, protein binding or nucleic acid hybridizations, and array washing, are very well known. Drying may be accomplished using any suitable drying method and conditions which will not decompose the probes and their bound targets, such as any suitable one or more of: air drying at room temperature or raised temperature; reduced pressure; centrifuging; or exposure to a dry unreactive gas stream (such as dry nitrogen).

In the case of a NO determination, the sample exposed array is then associated (580) with the test request(s), such as by the array unit 15b and medium 364 both then being placed in package 340 (optionally along with portable storage medium 324 if present) which package 340 is then forwarded to a reader station 90.

If the answer to the determination (564) is YES, then following exposure (570) of the sample to the array, features outside any retrieved sub-array pattern are rendered incapable of providing signal data representative of sample component binding, using deactivator (power supply 372 under control of processor 220b) by any of the methods previously described. In this case the sample array may optionally be associated (590) with the test request(s) as previously described (optionally along with portable storage medium 324 if present) and package 340 forwarded to reader station 90. Note that in this case if there is only one test request the test requests may not be required by reader station 90 since it in effect only receives feature locations capable of producing a sample component binding signal (other feature locations having been rendered incapable of producing such a signal).

Referring particularly to FIG. 9, at the array reader station 90 the sample exposed array is received (610) in package 340 optionally with the test request(s) and optionally with portable storage medium 324. The array identifier is read (620) using reader 226c and a determination (630) is made as to whether the received array has features which have already been rendered incapable of producing signal data representative of sample component binding. This determination may be made based on assumption (that is, the array unit is of a type which supports such rendering in typical user stations such as station 400 so it will be assumed to have been done). Alternatively, it may be made based on an express indication of such on the associated medium 364 received from user station 400. In a further alternative, it may be made based on the nature of signal data acquired from array feature locations at station 90 itself (for example, if there are a large number of features having no signal at all it may be assumed that rendering of those features has previously occurred at user station 400 but one would have to eliminate the possibility that this is not being caused by other factors such as a defective array or poor sample quality). In any event, if the answer to determination (630) is YES, a determination (634) is then made if an associated test request was received and, if so, which will be used. For example, even though the test request may not be needed in this situation it may be considered desirable to use it anyway to further ensure no array features outside all sub-array patterns retrieved at user station 400 are inadvertently read.

If there is no received associated test request and/or it is decided it is not needed (a NO answer to determination 634) signal data may be acquired and saved (640) from all array features and a same signal processing method applied (650) to all array features. In this situation the signal processing method may be relied upon to eliminate signal data from features previously rendered incapable (for example, they have no significant signal or are saturated where the processing method eliminates such features from any further consideration). Thus, signal data of sample component binding from feature locations irrelevant to any requested test has already been eliminated at user station 400 and cannot be recovered by anyone.

If there is a received associated test request (a YES to determination 634) then the method proceeds the same as in the case of a NO answer to determination (630). That is, the associated test request(s) is read (670) and one or more sub-array patterns for the test request(s) are retrieved (680) by processor 220c. The foregoing reading and retrieval may be by any of those methods already described in connection with user station 400. At this point it is determined (690) whether the array has feature locations outside of any or all retrieved sub-array patterns which can and will be rendered incapable of producing signal data representative of sample component binding, in the array reader 90 such as by label bleaching as already describe above.

If the answer to determination (690) is NO, then signal data is acquired and saved (700) only from the sub-array patterns retrieved at reader station 90, either by being not acquired or not saved as described above. Since data from irrelevant features has now been excluded by the foregoing event, a same signal processing method can then be applied (710) to all saved data for a given retrieved sub-array pattern. In this manner signal data from features irrelevant to any requested test is not saved (although it could be obtained in the future by again reading the array). However, different signal processing methods can be applied to saved data for different retrieved sub-array patterns. For example, as mentioned above the same signal processing method may be an encryption method based on a key, and an encryption method based on a different key is applied to signal data acquired from feature locations of a different retrieved sub-array pattern or also to feature locations outside any retrieved sub-array pattern. In this manner access to different results can be readily controlled by providing to an individual only the key(s) to results from one or more sub-arrays as desired. The individual may then use a decryption process which permits signal data from sub-array patterns to be decrypted based on distinct keys for distinct sub-array patterns (thus, knowing a key to one sub-array pattern does not permit recovery of signal data from a different sub-array pattern).

If the answer to determination (690) is YES, then such irrelevant signal data is eliminated by rendering the predetermined pattern of feature locations incapable of producing signal data representative of sample component binding, such as by label bleaching previously described. Again, in this situation signal data representative of sample component binding at feature locations irrelevant to any test requested, cannot later be recovered by anyone. In this situation signal data may be acquired and saved (730) from all feature locations of the read array. A same signal processing method may then be applied to acquired and saved signal data from all array feature locations since again data from irrelevant feature locations has been eliminated.

Signal processing methods which may be applied as described above, include feature extraction which can be performed using known methods, or those such as described in U.S. patent application Ser. No. 10/077,446 titled “Method And System For A Range Of Automatic, Semi-Automatic, And Manual Grid Finding During Feature Extraction From Molecular Array Data”, and Ser. No. 09/589,046 “Method And System For Extracting Data From Surface Array Deposited Features”, incorporated herein by reference. Following or before feature extraction, details of the array layout can be retrieved using the read array identifier 356 in a manner similar to that described in U.S. Pat. No. 6,180,351. Any results of methods of the present invention may then be used to make an assessment if one or more targets is present in a sample to which the array was exposed, or whether an organism from which the sample was obtained exhibits a particular condition (for example, cancer). The processed results may be further forwarded or transmitted to a remote location at which they are received, and can be re-transmitted to elsewhere from that location as desired.

Other methods of handling array data can be used as disclosed in U.S. Patent Application titled “METHOD AND SYSTEM FOR GENERATING VIRTUAL-MICROARRAYS”, filed by Paul Wolber on the same day as the present application and assigned to Agilent Technologies, Inc. (attorney docket number 10020348-1). The foregoing application is incorporated by reference into the present application.

Various and modifications to the particular embodiments described above are, of course, possible. Accordingly, the present invention is not limited to the particular embodiments described in detail above.

1. A method of using a chemical array unit having a chemical array with probes at multiple feature locations, comprising:
reading a request for a test which uses a sub-array of the array;
retrieving a pattern of the sub-array from a memory using the test request, which memory carries a pattern for the sub-array which is retrievable with the different test request.

2. A method according to claim 1 wherein the memory carries multiple sub-array patterns for the array each of which is retrievable with a different test request.

3. A method according to claim 1 additionally comprising reading an array identifier associated with the chemical array unit, and wherein the sub-array pattern is retrieved from the memory using both the array identifier and the test request, which memory carries multiple sub-array patterns for each of multiple arrays each sub-array pattern retrievable with a different combination of array identifier and test request.

4. A method according to claim 3 wherein the array unit carries the array identifier.

5. A method according to claim 1 wherein the array has been exposed to a sample, the method additionally comprising reading the chemical array and wherein signal data representative of binding of a sample component is not acquired and saved from feature locations outside any retrieved sub-array pattern.

6. A method according to claim 1 wherein the array has been exposed to a sample, the method additionally comprising reading the chemical array and wherein one or more of:
(a) signal data representative of binding of a sample component is acquired and saved from feature locations based on one or more retrieved sub-array patterns; or
(b) a same signal processing method is applied to acquired signal data representative of binding of a sample component from feature locations based on one or more retrieved sub-array patterns.

7. A method according to claim 6 wherein the test request is associated with the array.

8. A method according to claim 7 wherein the array unit carries an array identifier and the test request is associated with the array identifier.

9. A method according to claim 6 additionally comprising:
reading an array identifier associated with the chemical array unit, and wherein the sub-array pattern is retrieved from the memory using both the array identifier and test request, which memory carries multiple sub-array patterns for the array each retrievable with a different combination of array identifier and test request.

10. A method according to claim 9 wherein the array identifier and test request are associated with the array.

11. A method according to claim 7 wherein:
multiple requests for tests associated with the array are read, each of which uses a different sub-array of the array;
patterns of the sub-arrays are retrieved from memory using both the array identifier and the test requests, which memory carries multiple sub-array patterns each retrievable with a different combination of array identifier and test request.

12. A method according to claim 6 wherein the method comprises the acquiring and saving signal data representative of binding of a sample component from feature locations based on one or more retrieved sub-array patterns.

13. A method according to claim 12 wherein feature locations outside any retrieved sub-array pattern are incapable of providing signal data representative of binding of a sample component.

14. A method according to claim 13 wherein the feature locations outside any retrieved sub-array pattern are incapable of providing signal data representative of binding of a sample component as a result of binding of a sample component thereto having been prevented.

15. A method according to claim 13 wherein:
signal data of feature locations in a sub-array is acquired from reading signal from a label at those feature locations; and
the feature locations outside any retrieved sub-array pattern are incapable of providing signal data representative of binding of a sample component as a result of having an excess of the label thereon or having a material thereon which prevents reading of signal data representative of binding of a sample component.

16. A method according to claim 15 wherein the label is a fluorescent label.

17. A method according to claim 13 wherein the feature locations outside any retrieved sub-array pattern are incapable of providing signal data representative of binding of a sample component as a result of probes at those feature locations having been damaged to prevent binding.

18. A method according to claim 17 wherein the probes at the feature locations outside any retrieved sub-array are damaged by cross-linking.

19. A method according to claim 17 wherein the probes at the feature locations outside any retrieved sub-array are damaged by having been cleaved from those feature locations.

20. A method according to claim 13 wherein:
signal data representative of binding of a sample component at feature locations within a sub-array is acquired from a label at those feature locations; and
the feature locations outside any retrieved sub-array are incapable of providing signal data representative of binding of a sample component as a result of the label thereon having been damaged to prevent signal data being obtained from the label.

21. A method according to claim 20 wherein the label is a fluorescent or chemiluminescent label, the method additionally comprising damaging the label by bleaching the label at the feature locations outside any retrieved sub-array.

22. A method according to claim 12 wherein the total feature locations of all retrieved sub-array patterns is less than all feature locations of the array.

23. A method according to claim 22 wherein:
signal data representative of binding of a sample component is acquired from array feature locations of each retrieved sub-array pattern by illuminating those locations with an interrogating light and detecting any light emitted in response to the interrogating light; and
feature locations outside any retrieved sub-array pattern are not illuminated with the interrogating light.

24. A method according to claim 12 wherein:
signal data representative of binding of a sample component is acquired from both feature locations within a retrieved sub-array pattern and feature locations outside any retrieved sub-array pattern; and
acquired signal data representative of binding of a sample component from the feature locations within a retrieved sub-array pattern is saved in a memory while acquired signal data representative of binding of a sample component for the feature locations outside any sub-array pattern is not saved in the memory.

25. A method according to claim 6 wherein the method comprises applying a same signal processing method to acquired signal data representative of binding of a sample component from feature locations based on one or more retrieved sub-array patterns.

26. A method according to claim 25 wherein the same signal processing method comprises an encryption method based on a key, the method additionally comprising applying an encryption method based on a different key to signal data representative of binding of a sample component acquired from feature locations outside any retrieved sub-array pattern.

27. A method according to claim 25 wherein the signal processing method comprises a feature extraction method.

28. A method according to claim 27 wherein no feature extraction method is applied to feature locations outside any retrieved sub-array pattern.

29. A method according to claim 25 wherein:
multiple requests for tests associated with the array are read, each of which uses a different sub-array of the array; and
patterns of the sub-arrays are retrieved from memory using both the array identifier and the test requests, which memory carries multiple sub-array patterns each retrievable with a different combination of array identifier and test request.

30. A method according to claim 29 wherein:
different signal processing methods are applied to the acquired signal data from features of different retrieved sub-array patterns.

31. A method according to claim 30 wherein the test requests are associated with the array.

32. A method according to claim 30 wherein results from applying the different signal processing methods to acquired signal data representative of binding of a sample component from different sub-arrays, are independent such that a result from one sub-array cannot be derived from a result from one or more other sub-arrays.

33. A method according to claim 30 wherein results from applying the different signal processing methods to acquired signal data representative of binding of a sample component from the different patterns are forwarded to different locations.

34. A method according to claim 1 wherein the array has been exposed to a sample obtained from an individual and wherein the sub-array pattern is retrieved also using an identification of the individual.

35. A method according to claim 30 wherein results from applying some of the different signal processing methods to acquired signal data from the different sub-arrays, are rejected based on a comparison of the results or a comparison of a characteristic of the feature locations in the different sub-arrays.

36. A method of reading a chemical array unit having a chemical array with probes at multiple feature locations and which has been exposed to a sample, the method comprising reading the array wherein feature locations have been rendered incapable of providing signal data representative of binding of a sample component.

37. A method of using a chemical array unit having a chemical array with probes at multiple feature locations, comprising rendering a predetermined pattern of feature locations incapable of providing signal data representative of binding of a sample component.

38. A method according to claim 37 wherein the array has been exposed to a sample, the method comprising:
acquiring signal data from feature locations which have not been rendered incapable of providing signal data representative of binding a sample component.

39. A method according to claim 38 wherein:
signal data from feature locations is acquired from a label at those feature locations; and
rendering feature locations incapable of providing signal data representative of binding of a sample component comprises damaging a label at those feature locations to prevent signal data being obtained from the label.

40. A method according to claim 38 wherein the predetermined pattern of feature locations rendered incapable of providing signal data representative of binding a sample component consists of less than all the feature locations.

41. A method according to claim 38 wherein the rendering and the acquiring are executed in a same apparatus.

42. A method according to claim 41 wherein the rendering and the acquiring are executed while the array unit remains seated in a same holder.

43. A method according to claim 37 additionally comprising:
exposing the array to a sample.

44. A method according to claim 43 wherein the rendering a predetermined pattern of feature locations incapable of providing signal data representative of binding a sample component is performed before or during exposing the array to the sample.

45. A method according to claim 37 additionally comprising:
reading a request for a test which uses a sub-array of the array; and
retrieving a pattern of the sub-array from a memory using the test request, which memory carries multiple sub-array patterns for the array each retrievable with a different test request;
wherein:
the predetermined pattern of feature locations rendered incapable of providing signal data representative of binding of a sample component, comprises feature locations outside any retrieved sub-array pattern.

46. A method according to claim 37 wherein the rendering comprises selectively preventing binding of a sample component to probes at those feature locations.

47. A method according to claim 46 wherein the selectively preventing comprises activating heating elements at some of the feature locations.

48. A method according to claim 37 wherein:
a detectable signal is provided by a label which is bound to feature locations at which a sample component is bound to probes; and
the rendering comprises providing an excess of the label at those features.

49. An apparatus for use with a chemical array unit having a chemical array with probes at multiple feature locations, comprising:
an interrogating source;
a detector to detect signal generated in response to the interrogating source; and
a processor which causes the apparatus to execute a method of claim 1.

50. An apparatus for use with a chemical array unit having a chemical array with probes at multiple feature locations, comprising:
a light source to illuminate array feature locations with an interrogating light, which light source may or may not be the same as the light source of a deactivator;
a detector to detect light emitted in response to the interrogating light; and
a processor which causes the apparatus to execute a method of claim 2.

51. An apparatus for use with a chemical array unit having a chemical array with probes at multiple feature locations, comprising:
a deactivator which renders feature locations incapable of providing signal data representative of binding of a sample component; and
a processor controlling the deactivator so as to execute a method of claim 35.

52. An apparatus according to claim 51 wherein the processor additionally retrieves the pattern of feature locations to be rendered incapable of providing signal data representative of binding of a sample component, from the memory using the test request.

53. An apparatus according to claim 52 wherein the deactivator comprises a power supply controlled by the processor so as deliver power to selected heating elements at array feature locations in accordance with the pattern.

54. An apparatus according to claim 52 wherein the deactivator comprises a light source.

55. An apparatus according to claim 54 wherein the apparatus additionally comprises:
a light source to illuminate array feature locations with an interrogating light, which light source may or may not be the same as the light source of the deactivator; and
a detector to detect light emitted in response to the interrogating light

56. A computer program product comprising a computer readable medium carrying a computer program which when loaded into a computer executes a method of claim 1.

57. A computer program product comprising a computer readable medium carrying a computer program which when loaded into a computer executes a method of claim 6.

58. A computer program product comprising a computer readable medium carrying a computer program which when loaded into a computer executes a method of claim 37.

59. A method comprising retrieving a sub-array pattern of a chemical array from a memory using a test request, which memory carries a sub-array pattern for the array retrievable with a test request.

60. A method according to claim 59 wherein the memory carries multiple sub-array patterns for the array each retrievable with a different test request.

61. A method according to claim 59 wherein the sub-array pattern is retrieved from the memory using both an array identifier and the test request, which memory carries multiple sub-array patterns for each of multiple arrays, each pattern retrievable with a different combination of array identifier and test request.

62. A method according to claim 61 wherein the array identifier and test request are received from a remote location, and the retrieved pattern of less than all the array feature locations is communicated to the remote location.

63. A computer program product comprising a computer readable medium carrying a computer program which when loaded into a computer executes a method of claim 59.
